(19) United States
(12) Patent Application Publication
(10) Pub. No.: US 2016/0278827 A1
(43) Pub. Date: Sep. 29, 2016

(54) DYNAMIC OSTEOTOMY PLATE INCLUDING DEVICES, APPARATUS AND METHODS USING SUCH A PLATE

(71) Applicant: ESPMEN - CONSULTORIA, UNIPESSOAL, LDA, Porto (PT)

(72) Inventors: Joao Duarte COELHO DO SAMEIRO ESPREGUEIRA MENDES, Porto (PT); Antonio Jose CAETANO BAPTISTA, Porto (PT); Augusto Duarte CAMPOS BARATA DA ROCHA, Porto (PT); Diana Rebecca ESTEVES CARDOSO GAVAZZO RODRIGUES, Aveiro (PT); Helder MIGUEL PEREIRA, Touguinho (PT); Joao Paulo GERALDES TOURO PEREIRA, Porto (PT); Jorge Manuel TEIXEIRA DA SILVA, Porto (PT); Rogerio BARBOSA PEREIRA, Mazarefes (PT)

(73) Assignee: ESPMEN - CONSULTORIA, UNIPESSOAL, LDA, Porto (PT)

(21) Appl. No.: 14/778,255

(22) PCT Filed: May 12, 2014
(86) PCT No.: PCT/IB2014/061383

§ 371 Date: Sep. 18, 2015

(30) Foreign Application Priority Data
Mar. 19, 2013 (PT) 106844

(51) Int. Cl. A61B 17/80

(52) U.S. Cl. CPC A61B 17/8061; A61B 17/8095

(57) ABSTRACT

Featured are a device, a surgical kit and an osteotomy method for aligning a bone or bony structure. Such a method includes providing a base member that extends in at least two directions and spans the cut in the bone; a securing mechanism mechanically coupled to the base member for securing the base member to the bone; and an adjusting mechanism coupled to the base member and including at least one first portion. Such a method includes securing one end of the base member to the bone using the securing mechanism and rotating the at least one first portion so it contacts at least one of the opposing surfaces of the cut thereby maintain the two opposing surfaces in fixed spaced relation. Such a method also includes securing the base member's other end to the bone using the securing mechanism.

### FIELD OF INVENTION

The present invention generally relates to devices, apparatus, kits and methods for use in connection with Osteotomy surgical procedures and more particularly to such devices, apparatus, kits and methods for use in connection with high tibial surgical procedures.

### BACKGROUND OF THE INVENTION

An osteotomy is a surgical operation or procedure whereby a bone or bony structure is cut to shorten, lengthen, or change its alignment. It is sometimes performed to correct a hallux valgus, or to straighten a bone that has healed crookedly following a facture. It is also used to correct a coxa vara, genu valgum and genu varum. The operation is typically done with the patient under a general anesthetic. Osteotomy also is one method to relieve pain in arthritis, especially of the hip and knee. It also is not uncommon to see the Osteotomy being replaced later by means of a joint replacement in an older patient.

It should be recognized that due to the serious nature of this procedure, recovery may be extensive. Careful consultation with a physician is important in order to ensure proper planning during a recovery phase. Tools exist to assist recovering patients who may have non weight bearing requirements and include bedpans, dressing sticks, long-handled shoe-horns, grabbers/reachers and specialized walkers and wheelchairs.

In addition to such post-operative concerns, there is a need for extensive and detailed pre-planning by the surgeon to assess the condition of the knee or hip and to determine the kind and extent of the cut to be made in the bone or bony structure before performing the procedure.

One high tibial osteotomy (HTO) method or technique that is used frequently requires a wedge shaped cut be made in the bone or bony structure from the side of the tibia which does not go completely through or across the bone or bony structure. After cutting the wedge, the surgeon typically places a wedge shaped graft in the wedge shape opening, this wedge shaped graft can be obtained by harvesting bone from the patient's skeletal structure, it can be bone harvested from a cadaver or it can be made from a bio compatible material such as PEEK.

After installing this wedge shaped graft or body in the wedge shaped cut, the surgeon then locates a plate member across the bone or bony structure such that it bridges or spans the wedge shaped cut. The plate member typically includes a plurality of through holes on opposing sides of the plate member. The surgeon then secures the plate member to the bone or bony structure by passing a fastener through each of these through apertures and securing each fastener to the bone or bony structure. The plate and screws initially stabilize the bone and bony structure until, the wedge shaped cut is filled by bone growth. After the bone ingrowth fills in the wedge shaped cut, the surgeon could later remove the plate and fasteners from the patient. However, it is not uncommon for the plate and fasteners to remain in place so as to avoid further surgery.

High tibial osteotomy (HTO) is a surgical procedure used to change the mechanical weight-bearing axis and alter the loads carried through the knee (Rodner C M, Adams D J, Diaz-Doran V, et al. Medial opening wedge tibial osteotomy and the sagittal plane: the effect of increasing tibial slope on tibiofemoral contact pressure). The American journal of sports medicine 2006; 34:1431-41). Conventional indications for HTO are medial compartment osteoarthritis and symptomatic varus malalignment of the knee. Formerly, knee instability associated with varus thrust was been considered a contraindication. However, today the indications include patients with chronic ligament deficiencies and malalignment, because an HTO procedure can change not only the coronal but also the sagittal plane of the knee (Kimura Y, Ishibashi Y, Tsuda E, Fukuda A, Tsukada H. Sagittal realignment osteotomy for increased posterior tibial slope after opening-wedge high tibial osteotomy: a case report. Sports medicine, arthroscopy, rehabilitation, therapy & technology: SMARTT 2009; 1:26; Yim J H, Seon J K, Song E K. Posterior tibial slope in medial opening-wedge high tibial osteotomy: 2-D versus 3-D navigation. Orthopedics 2012; 35:60-3).

Scarce information can be found in the literature considering the effect of HTO in the sagittal plane. However, its modification has a significant impact on biomechanics and joint stability (Kimura Y, Ishibashi Y, Tsuda E, Fukuda A, Tsukada H. Sagittal realignment osteotomy for increased posterior tibial slope after opening-wedge high tibial osteotomy: a case report. Sports medicine, arthroscopy, rehabilitation, therapy & technology: SMARTT 2009; 1:26). Indeed, decreased posterior tibial slope causes posterior tibia translation and helps the anterior cruciate ligament (ACL)-deficient knee. Vice versa, increased tibial slope causes anterior tibia translation and helps the posterior cruciate ligament (PCL)-deficient knee. In selected cases soft tissue procedures alone are often unsatisfactory for chronic posterior instability if alignment is not corrected. Since limb alignment is the most important factor to consider in lower limb reconstructive surgery, diagnosis and treatment of limb malalignment should not be ignored in management of chronic ligamentous instabilities (Savarese E, Bisicchia S, Romeo R, Amendola A.

Role of high tibial osteotomy in chronic injuries of posterior cruciate ligament and posterolateral corner. Journal of orthopaedics and traumatology: official journal of the Italian Society of Orthopaedics and Traumatology 2011; 12:1-17).

Referring now to EP Patent 2496161 and WO Patent 055353 there is found a mechanical device intended for correcting a malformation of the bones of the body. The device includes a top plate suitable for sliding over a bottom plate by means of a guide. The plates can be screwed on either side of an angled notch made in the bone. The plates comprise respectively top and bottom bearings of the notch of the bone. The bearings form an acute-angle bevel which substantially matches the surfaces of the notch. One of the plates is provided with an adjustment screw for continuous adjustment of the relative positions of the plates. The device also comprises a locking screw suitable for maintaining the open position of the device and, consequently, the final angle of the notch of the bone in the desired position. The top bearing of the notch of the bone having angular adjustment is suitable for correcting the angle of the beveled surface of the bearing, in particular the tibial slope, by means of a tapered screw adjusting an angular space in the bearing.

Referring now to Mexican Patent 2009/003259 there is found an inner fixing plate that is fixed with two distal screws, where the anatomical design thereof allows the same to be easily located; it is also a stable system which allows rehabilitation to be carried out without requiring an external immobilization cast. The purpose of this invention is to provide a metaphyseal plate totally different from those currently available on the market, which improves the results in surgery (valgus osteotomy), upon ensuring, with the stability and fixing strength thereof, the bone wound healing, thus enabling an early and quick rehabilitation.

Referring now to EP Patent 2086434 there is found an osteotomy plate for maintaining the spacing of a wedge-like opening in bone, the osteotomy plate includes: a body having a front side and a back side; a protrusion extending out of the back side of the body for disposition in the wedge-like opening; and a plurality of mounting holes for receiving fixation screws therein, the mounting holes being formed in the body such that when the protrusion is disposed in the wedge-like opening, the mounting holes direct the fixation screws into bone on either side of the wedge-like opening.

Referring now to EP Patent 2068784 there is found an osteotomy plate for maintaining the spacing of a wedge-like opening in bone, the osteotomy plate having a body having a front side and a back side; a key extending out of the back side of the body for disposition in a keyhole formed in the bone along the wedge-like opening; and a plurality of mounting holes for receiving fixation screws therein, the mounting holes being formed in the body such that when the key is disposed in the keyhole, the mounting holes direct the fixation screws into bone on either side of the wedge-like opening.

Referring now to Korean Patent 2008/1020080107390 and EP Patent 1986557 there is found an improved tibial plateau leveling osteotomy plate. The plate is contoured in its proximal head portion to more closely resemble the structure of the tibial bone segment that is cut and rotated during the procedure. The plate also preferably has screw holes in the proximal head portion that are machined through the pre-contoured proximal head portion and are designed to angle the screw in a targeted screw path with respect to the osteotomy.

Referring now to PCT/WO Patent 2008/039508 there is found an osteotomy plate for maintaining the spacing of a wedge-like opening in bone, the osteotomy plate comprising: a body having a front side and a back side; a protrusion extending out of the back side of the body for disposition in the wedge-like opening; and a plurality of mounting holes for receiving fixation screws therein, the mounting holes being formed in the body such that when the protrusion is disposed in the wedge-like opening, the mounting holes direct the fixation screws into bone on either side of the wedge-like opening.

Referring now to PCT/WO Patent 2008/019114 there is found an osteotomy plate for maintaining the spacing of a wedge-like opening in bone, the osteotomy plate having a body having a front side and a back side; a key extending out of the back side of the body for disposition in a keyhole formed in the bone along the wedge-like opening; and a plurality of mounting holes for receiving fixation screws therein, the mounting holes being formed in the body such that when the key is disposed in the keyhole, the mounting holes direct the fixation screws into bone on either side of the wedge-like opening.

Referring now to PCT/WO Patent 2007/100513 there is found a tibial plateau leveling osteotomy plate is disclosed. The plate is contoured in its proximal head portion to more closely resemble the structure of the tibial bone segment that is cut and rotated during the procedure. The plate also preferably has screw holes in the proximal head portion that are machined through the pre-contoured proximal head portion and are designed to angle the screw in a targeted screw path with respect to the osteotomy.

Referring now to Korean Patent 2006/100641312 there is found a metallic fixing apparatus for opening a wedge in high tibial osteotomy is provided to obtain stable fixation without causing the damage of soft tissues by reducing volume thereof.

Referring now to PCT/WO Patent 2006/092461 there is found a device for guiding a cutting tool, which is intended, in particular, for use during a canine tibial plateau levelling operation. The inventive device comprises: a base plate and a support plate which are fixed together to the tibia; and a positioning mechanism which is fixed to the support plate, said mechanism being configured such that the tool can only move along the desired cutting trajectory. One configuration of the positioning mechanism includes a rotationally-mounted compass which can be used to make cylindrical incisions with precision. Once the corresponding incisions have been made, the tibia superior segment can be rotated until the tibial plateau has been levelled and the superior and inferior segments can be fixed using a compression plate.

Referring now to PCT/WO Patent 2002/098306 there is found a surgical device comprising a plate member for mounting to a bone of a subject. The plate member has a first end, a second end, a first face and a second face. At least one spacer member is adapted to be mountable to at least a portion of the plate member and relatively moveable towards and/or away from its first end. The spacer member extends outwardly from the second face of the plate member and is prevented from moving away from the first end by a stop member positioned between the first and second ends of the plate member.

Referring now to PCT/WO Patent 1996/024295 there is found a surgical kit for performing a tibial osteotomy, including a pair of mounting pins for attaching an osteotomy guide in a predetermined relation to a tibia; wherein the osteotomy guide includes a transverse slot, defining a transverse cutting plane, adapted to receive and guide a transverse cutting blade for making a transverse cut into the tibia, and a plurality of oblique slots angularly offset from the transverse slot, each oblique slot defining an oblique cutting plane, adapted to receive and guide an oblique cutting blade for making a selected oblique cut into the tibia, wherein the intersection of each oblique cutting plane with the transverse cutting plane defines a wedge of bone which may be removed from the tibia. Also included with the surgical kit are a compression clamp adapted to apply compressive forces to a first portion of the tibia above the transverse cut and to a second portion of the tibia below the oblique cut to draw the first and second portions together, and a fixation plate adapted to hold the portions of the tibia together during healing. A method for performing an upper tibial osteotomy is also provided which employs the osteotomy surgical kit.

Referring now to EP Patent 0095296 there is found a guide assembly for use in a tibial osteotomy wherein two pairs of parallel guide pins are inserted into the tibia at a predetermined angle with respect to each other through a guide block. The adjacent surfaces of the pairs of pins can then be used to precisely guide a saw by which a wedge-shaped segment of the tibia is removed preferably using a guide plate.

It thus would be desirable to provide a new device, system, surgical kit and methods for osteotomy method for aligning a bone or bony structure. It would be particularly desirable to provide such devices, surgical kits and methods that would utilize such a plate and related structure to adjust the wedge shaped gap and the angle of the gap as well as stabilizing the wedge shaped gap in comparison to prior art devices. More specifically, it would be further desirable to provide such a device, kit and methods that allows for dynamic correction, i.e., accurate and comprehensive mechanical realignment in frontal, sagittal and in sagittal and frontal planes simultaneously during osteotomies. Such devices and kits preferably would be no more complex in construction and costly than prior art devices and such methods would not require highly skilled users to utilize the device and kit as compared to conventional methods and techniques.

### SUMMARY OF THE INVENTION

The present invention features a device, a surgical kit, a system and methods for surgically aligning bone or bony structure using osteotomy techniques in which bone or bony structures are cut such as to enable the formation of a wedge shape, but that does not go through the bone or bony structure. In other words, the bone or bony structure retains a hinge or hinge like structure.

In more particular aspects, the present invention features, a dynamic osteotomy plate which enables correction, i.e., accurate and comprehensive mechanical realignment in frontal, sagittal and in sagittal and frontal planes simultaneously during osteotomies. High tibial open-wedge osteotomy (HTO) with step-plates (Puddu-like systems) or locking compression plates are used to increase stability and provide control in frontal plane.

The developed dynamic osteotomy plate includes specific cams' slots and a full set of snail cams or spiral cam like structures with incremental variation in which different heights can be chosen by cams' rotation, adapted and blocked in order to maintain a uniplanar or biplanar wedge-like opening in the bone. The plate has a contoured body for anatomic adaptation to tibial proximal metaphyses (with front and back sides) and two lateral cams' slots which are both compatible with different or equal cams sizes, being these independently adjustable within their broad range of heights. Thus, the cam's slots can receive matched or unmatched cams, in particular in its range of heights or geometry. Every cam has a cut on its brim which in association with double slot morphology and slight cam rotation after its placing, only possible in a unique position, results in cam and plate slot concentricity and eliminates the risk of the cam falling out of the plate during bone implantation or rotation for height adjustment. The plate has two proximal and two distal holes for locking screw placement for fixation to long bone cortex.

The referred snail cams allow for a continuous (non-discrete) range of positions and sizes ranging for example from 6 up to 14 mm. The rotation of the cams provides a mechanism or means for uniplanar and/or biplanar step-effect in several degrees of inclination in frontal, sagittal or combined frontal and sagittal orientation. This way the cams ensure appropriate mechanical stability for bone fixation while enabling control of the objectives for the osteotomy defined in preoperative planning.

This osteotomy plate allows, intra-operatively, the full control of osteotomy position, in frontal, sagittal plane or frontal and sagittal planes simultaneously by choosing the proper step-plate for open-wedge in the frontal plane and then adapt it to specific patient demands in sagittal plane, i.e., it can reproduce anatomic slope; increase it in Posterior-Cruciate Ligament-deficient knees or decrease it in Anterior Cruciate Ligament-deficient knees. The disclosed osteotomy plate is particular suited for controlled tibial osteotomy and a special design is also provided for simultaneous HTO and ACL reconstruction (best suited for tibial tunnel placement required during ACL repair).

According to one aspect of the present invention, there is featured a device for aligning a bone or bony structure using an osteotomy technique in one or both of a frontal and/or sagittal planes. Such a device includes a base member, a securing mechanism, and an adjusting mechanism. The base member extends in at least two directions, one direction being set so the base member spans or bridges the cut in the bone or bony structure. Such a cut can be wedge shaped or other appropriate shape for the particular procedure to be performed. The securing mechanism is mechanically coupled to the base member and arranged to secure the base member to the bone or bony structure. In illustrative embodiments, the securing mechanism includes any of a number of mechanical fasteners as known to those skilled in the art and otherwise appropriate for the intended use.

The adjusting mechanism also is coupled to the base member and includes at least one first portion. Also the at least one first portion is arranged so as to extend into the cut proximal the end surface of the cut. Also, the at least one first portion is rotatable with respect to the base member and dimensioned so as to be capable of maintaining two opposing surfaces of the cut or wedge shaped cut in fixed spaced relation when disposed in the cut. In further embodiments, the adjusting mechanism includes two first portions that are spaced from each other, each first portion being rotatable with respect to the base member and each first portion being dimensioned so as to be capable of maintaining two opposing surfaces of the cut in fixed spaced relation.

In yet further embodiments, each of the first portions includes a spiral shaped member that is rotatable with respect to the base member. Such a spiral shaped member also is configured so that the radius of the spiral varies as a function of the arc of rotation. In addition, the radius of one spiral member varies differently as a function of the arc of rotation than the second spiral member, whereby the two spiral members can provide the device with two degrees of freedom. Further, each spiral shaped member can comprise a snail cam structure.

In yet further embodiments, each of the adjusting mechanism first portion includes a spiral shaped member that is rotatable with respect to the base member and which is configured so that the radius of the spiral varies as a function of the arc of rotation. Also, the radius of the spiral is established so as to define a plurality of discrete radii as a function of the arc of rotation.

In yet further embodiments, the base member is configured with a mating structure and the adjusting mechanism includes a complimentary mating structure that is arranged to mate with the base member mating structure so as to hold each adjusting mechanism portion in fixed relation with respect to the base member. In addition, the base member mating structure comprises a slot in the base member and the adjusting mechanism mating structure comprises a mating portion that is slidably received in the base member slot. Also, the adjusting portion mating portion can be arranged so that it is rotatably secured to the adjusting portion first portion.

In yet further embodiments, the base member includes a plurality of through apertures arranged so as to be located on either side of the cut and the securing mechanism includes a plurality of mechanical fasteners one for each through aperture, wherein one mechanical fastener passes through a respective through aperture and is secured to the bone or bony structure whereby the base member is secured to the bone or bony structure. Further, the base member is configured and arranged so as to include a plurality of through apertures arranged so as to be disposed opposite one side of the cut. Also, the base member can be configured and arranged so as to include a plurality of through apertures arranged so as to be disposed opposite both sides of the cut.

In yet further embodiment, the device is configured and arranged so as to used in a high tibial osteotomy procedure.

According to yet a further aspect of the present invention there is featured a surgical kit for aligning a bone or bony structure using an osteotomy technique in one or both of a frontal and/or sagittal planes. Such a surgical kit includes a base member extending in at least two directions, one direction being set so the base member spans the cut in the bone or bony structure and a plurality of adjusting mechanisms, each being arranged so as to be separately mechanically coupled to the base member, each including a spiral member that is arranged so as to extend into the cut proximal the end surface of the cut. Also, each spiral member is rotatable with respect to the base member and is configured so that the radius of the spiral varies as a function of the arc of rotation and so as to be capable of maintaining two opposing surfaces of the cut in fixed spaced relation when disposed in the cut. More particularly, the radius of one of the plurality of spiral member varies differently as a function of the arc of rotation than another of the plurality of spiral members.

In further embodiments, the surgical kit further includes 3 or more adjusting mechanisms with 3 or more spiral members. At least one of the spiral members is configured so the radius of one of the three or more spiral member varies differently as a function of the arc of rotation than other 2 spiral members. According to another embodiment, the surgical kit includes a plurality of pairs of adjusting mechanisms wherein the spiral members for one pair of the adjusting mechanisms are configured so the radius of said one pair member varies differently as a function of the arc of rotation for the spiral members of the other pair of plurality of adjusting mechanisms.

According to yet another aspect of the present invention there is featured an osteotomy method for aligning a bone or bony structure. Such a method includes providing: a base member extending in at least two directions, one direction being set so the base member spans the cut in the bone or bony structure; a securing mechanism that is mechanically coupled to the base member and arranged to secure the base member to the bone or bony structure; and an adjusting mechanism that is coupled to the base member and including at least one first portion.

Such a method also includes securing one end of the base member to the bone or bony structure using the securing mechanism and so the at least one portion extends into the cut proximal an end surface of the cut. Such a method also includes rotating the at least one first portion with respect to the base member so as to contact at least one of two opposing surfaces of the cut so as to thereby maintain the two opposing surfaces of the cut in fixed spaced relation. In addition, such a method includes using the securing mechanism and securing the other end of the base member to the bone or bony structure.

In further embodiments, such a osteotomy method further includes the step of cutting a and creating a wedge shaped opening in the bone or bony structure that wedge shaped opening extending partially through the bone or bony structure.

In yet further embodiments, such an osteotomy method is used in connection with a high tibial osteotomy procedure.

Other aspects and embodiments of the invention are discussed below.

### DEFINITIONS

The instant invention is most clearly understood with reference to the following definitions:

The term tissue when used herein shall be understood to include other parts or structure of a human body including, but not limited to cartilage, muscle, bone, bony structures (e.g., vertebrae) and ligaments.

USP shall be understood to mean U.S. Patent Number and U.S. Publication No. shall be understood to mean U.S. Published Patent Application Number.

### BRIEF DESCRIPTION OF THE DRAWING

For a fuller understanding of the nature and desired objects of the present invention, reference is made to the following detailed description taken in conjunction with the accompanying drawing figures wherein like reference characters denote corresponding parts throughout the several views and wherein:

FIG. 1 is an anterior view of the base member of the device of the present invention.

FIG. 2 is a posterior view of the base member of FIG. 1, the side of the base member that is in direct contact with the bone.

FIG. 3 shows a perspective view of the posterior of the base member, where the two cams are placed in the base member.

FIG. 4 is a perspective view of the anterior of the base member.

FIG. 5 is a lateral or side view of the base member.

FIGS. 6A-C are three views of identical cams that can be placed in the implant/device of the present invention; a perspective view (FIG. 6A), an anterior view (FIG. 6B) and a posterior view (FIG. 6C).

FIGS. 7A-C shows three views of locking member; a perspective view (FIG. 7A), an anterior view (FIG. 7B) and a posterior view (FIG. 7C).

FIGS. 8A-C are perspective views showing three distinct cam sizes.

FIG. 9 shows an anterior perspective view of the base member and cams, where the insertion of the two cams laterally in the base member is illustrated.

FIG. 10 shows an anterior perspective view of the base member and inserted cams.

FIG. 11 shows an anterior perspective view of the base member and inserted cams, after the left cam has been rotated.

FIG. 12 is another anterior perspective view of the base member, inserted and rotated cams and the locking member, illustrating the placing of the locking member.

FIG. 13 is an anterior perspective view of the base member, with the cams secured by the placed locking member or locking lid.

### DESCRIPTION OF THE PREFERRED EMBODIMENT

The present invention relates to a biomechanical implant device that relates to the surgical procedure of osteotomy, including high tibial osteotomy (with all its purposes) but this is not limiting as it is also contemplated that the device, surgical kit and methodology of the present invention also can be used or adapted for us in connection with distal femur osteotomy, radius osteotomy, distal tibia osteotomy and other procedures. As further described herein, the present invention allows for a simple, safer and more flexible implant device than currently available

The plate or base member as herein described yields a novel High Tibial Osteotomy (HTO) implant and bone alignment correction mechanism. It contemplates or comprehends cases of varus knee, and may be inserted temporarily in younger patients or permanently in older patients. In other words, in some cases the implant may be inserted, and once the bone has been corrected and grown, the implant may be removed; whilst in other cases the implant may simply be left attached to the bone indefinitely.

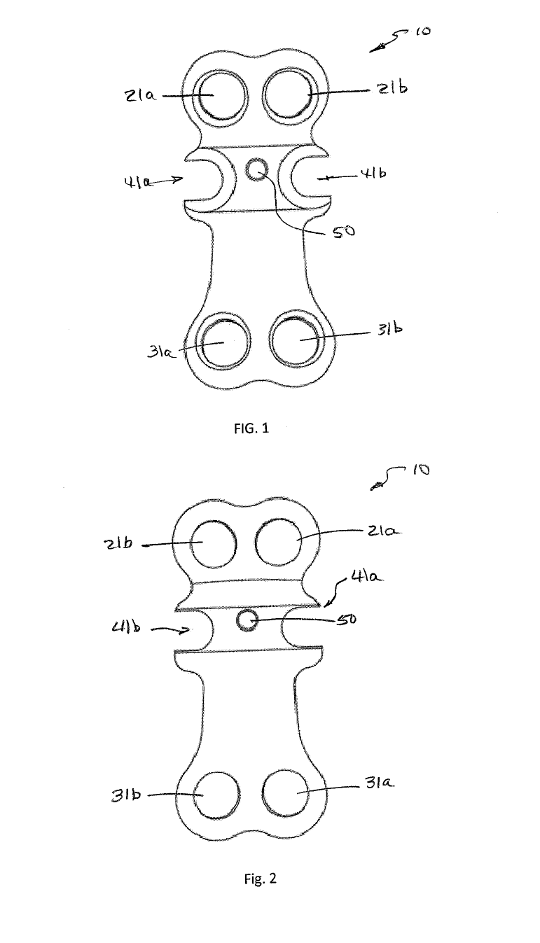

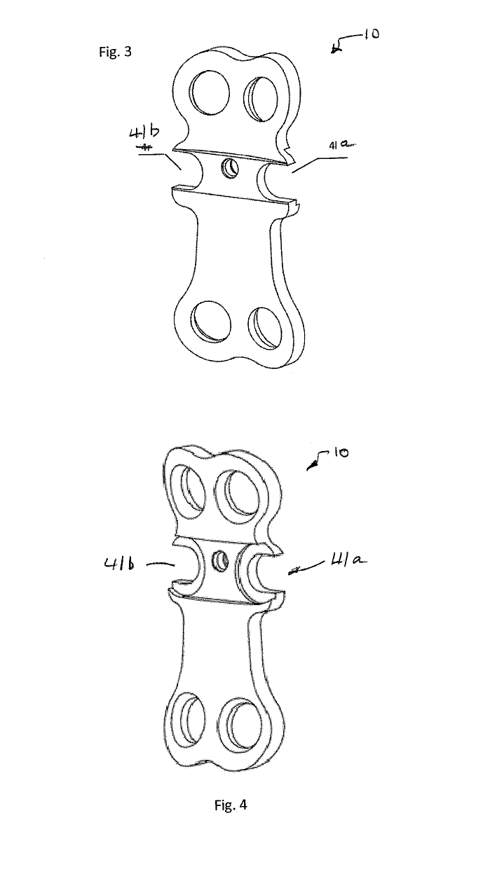

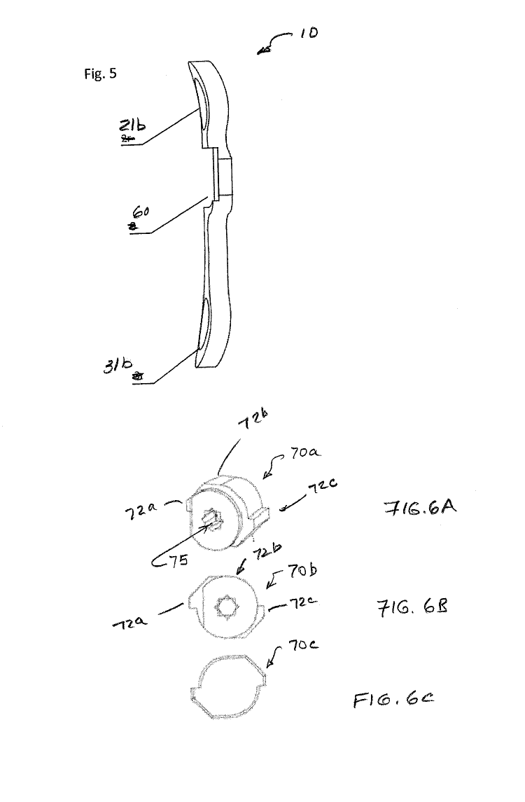

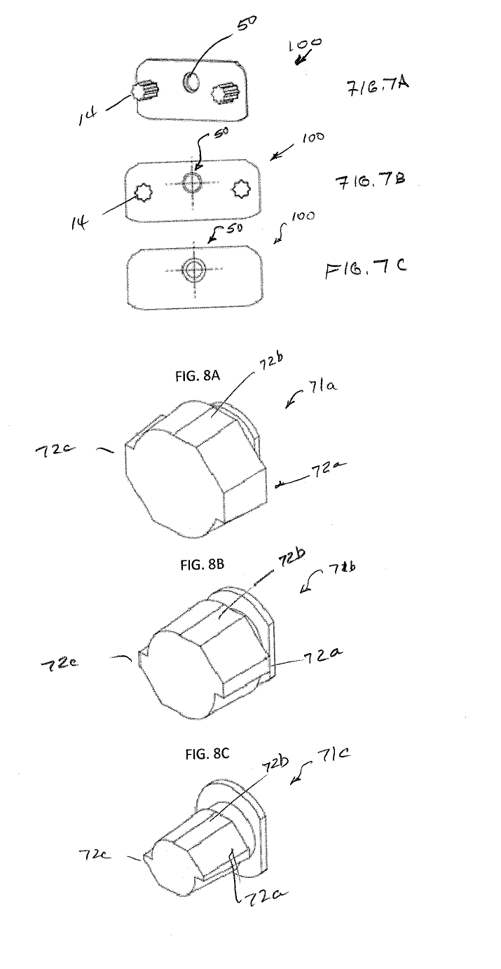

Referring now to the various figures of the drawing wherein like reference characters refer to like parts, there is shown in FIGS. 1-5 various views of a base member 10 or base plate according to the present invention, where FIG. 1 is an anterior view of the base member; FIG. 2 is a posterior view of the base member (i.e., the side of the base member that is in direct contact with the bone); FIG. 3 shows a perspective view of the posterior of the base member, where the two cams will be placed in the base member; FIG. 4 represents a perspective view of the anterior of the base member and FIG. 5 is a lateral view of the base member.

As illustrated, the base member 10, plate like member or base plate includes a plurality of through aperture or holes 21a,b; 31a,b configured for use with locking screws or other mechanical fasteners as are known in the art to secure the base member 10 to the bone or bony structure. More specifically, the base member includes two upper holes or through apertures 21a,b for locking screws and two lower holes or through apertures 31a,b.

The base member also includes two lateral slots 41a,b and a fifth hole or through aperture 50. As described herein, the two slots receive the two cams and the fifth through aperture 50 receives a screw therein after the locking member or locking lid has been fitted to the cams. Such a base member 10 is made of any of a number of bio-compatible materials as are known to those skilled in the art and otherwise appropriate for the intended use. Such materials include bio-compatible metals such as titanium and bio-compatible plastics such as PEEK. In further aspects, the side of the base member shown in the posterior view (FIG. 2) is the side of the base member that is in direct contact with the bone. In addition, FIG. 3 shows a perspective view of the posterior of the base member, whereat the two cams will be placed in the base member.

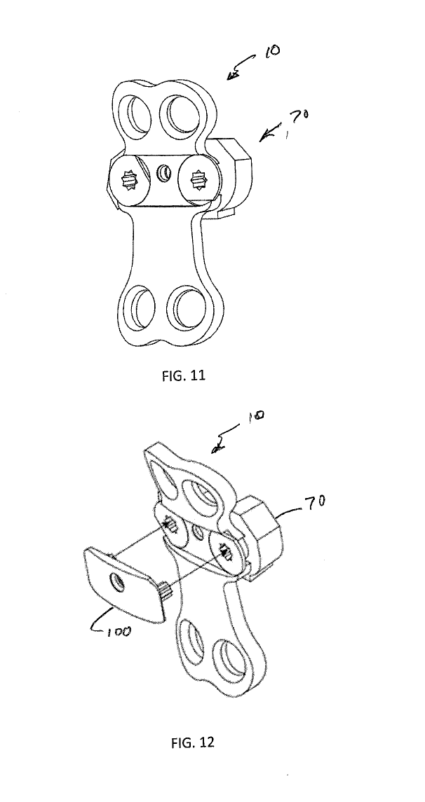

The lateral or side view of the base member 10 illustrated in FIG. 5 shows one upper and one lower locking screw holes 21b, 31b. Also shown is the indent 60 in the base member 10 corresponding to the slots 41a,b in which are received the cams 70 (FIG. 9) and the locking member 100 (FIG. 12) or locking lid.

Referring now to FIGS. 6A-C there are shown various views of two identical cams 70a,b that can be placed in the device/implant; a perspective view (FIG. 6A), an anterior view (FIG. 6B) and a posterior view (FIG. 6C). As indicated herein the cams 70a,b can be configured so that a portion of the cam (i.e., one of facets 72a-c) engages the bone or bony structure so as to maintain opposing surfaces of the cut or wedge shaped opening spaced from each other. A single cam allows for a certain range of angle for the aperture, opening or cut wedge. One snail cam or cam having a spiral member can serve for several different cases. However, when dealing with very different cases, different size snail cams may be necessary and which is embodied in the subject.

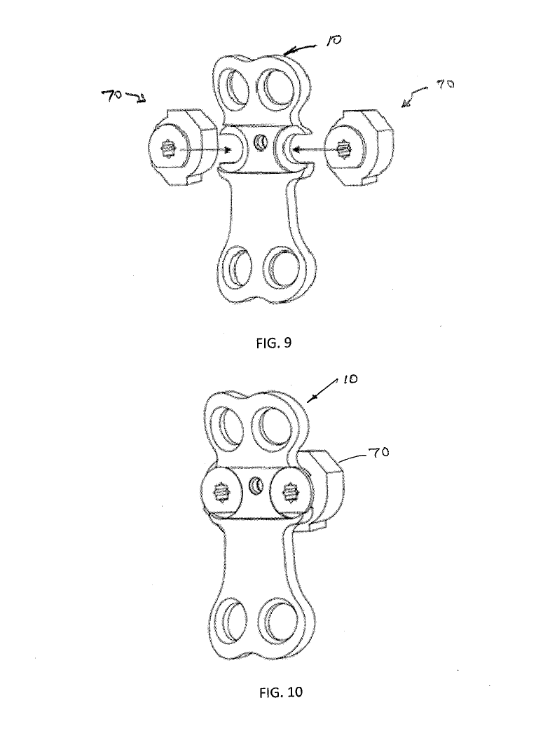

As more particualry shown, the outer surfaces of the cam are arranged so as to provide three facets or flat surfaces 72a-c which can contact or otherwise engages the bone or bony structure when the surgeon rotates the cam 70a. See also FIGS. 8A-C. Each of the surfaces or facets 72a-c are at a discrete and different radius or height from the axis of rotation and each surface/facet also are located at a discret angular position or arc of rotation. Thus, by rotating the cam to one of these angles or arcs of rotation, one of the surfaces can contact the bone or bony structure. In addition, the surgeon also can use these different surface so by rotating the cam they can adjust the cut or created opening as herein described in more detail. While these surfaces or facets are shown as being limiting this is not limiting as the outer surface can have any of a number of surface arrangements as are known in the art (e.g., continuous varying curver structure). It also should be recognized that the outer surface of this cam also forms a spiral like structure or spiral member because of the varying radii.

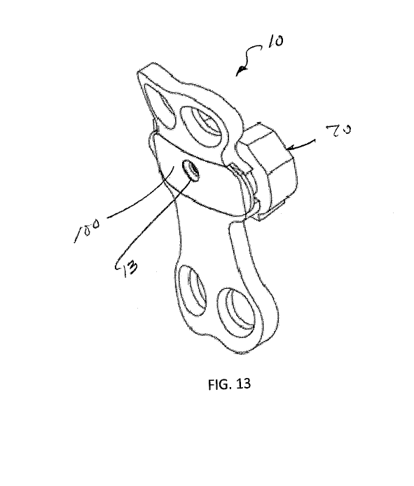

Referring now also to FIG. 9 there is shown an anterior perspective view of the base member 10 and cams 70, where the insertion of the two cams laterally in the base member is illustrated. Referring now also to FIG. 10 there is shown an anterior perspective view of the base member 10 and inserted cams 70. Referring now also to FIG. 11 there is shown an anterior perspective view of the base member 10 and inserted cams 70. More specifically, FIG. 11 illustrates the left cam after it has been rotated. Referring now also to FIG. 12 there is shown is another anterior perspective view of the base member 10, inserted and rotated cams 70 and the locking member 100, illustrating the placing of the locking member. Referring now also to FIG. 13 there is shown an anterior perspective view of the base member 10, with the cams 70 secured by the placed locking member 100 or locking lid.

In the present invention, all that varies from one sized snail cam to another sized snail cam is its height, all other cam characteristics are the same. Therefore, seeing as it's rare or even impossible for two cases to be exactly the same, with a few different cams all cases may be treated in the same way, just being necessary to take advantage from the full liberty of combining equal or different cams in size in the same implant. Thus, through their range of heights can be separately or simultaneously selected by simple turning the cam, multiplanar bone correction using the devices and methods of the present invention is feasible, fine tuning and time saver.

Depending on the surgeons' initial assessment on how wide the correction angle needs to be in different planes, the surgeon initially makes a prediction or determination of the necessary aperture for the angular correction in the bone. Once this is done the cams can be selected. So as to better explain how the implant is handled, it was stipulated that the posterior side of the implant is the side with the cams, whilst the anterior side is the side with the indentation 60 for the locking member 100 or locking lid and the one the surgeon sees once the implant has been inserted.

Once the snail cams 70 are placed (there is only one possible position for fitting the cam with the slot 41a,b due to the cut brim in the cam and in the two lateral slots 41a,b of the implant) and slightly rotated, not only is the concentricity between the cams and the plate slots a high likelihood if not certainty, but the risk of the cam 70 falling out of the base member 10 or base plate during bone implantation and cam rotation is eliminated. Then the implant with the cams 70 locked will be ready to be inserted in the bone as soon as a standard wire tool is in place on the implant. This step does not have the risk of the cams falling out since they are provided with a novel mechanism of safety, i.e., combined effect of cam's cut brim and slots' geometry.

At this point the surgeon can rotate the two snail cams 70, which innovative geometry allows multiple adjustable heights, in every case, coincident with flattened surfaces for reduced pressure bone contact. As before referred the two cam's slots are compatible with different or equal cam sizes and being independently adjustable within their broad range of heights. Thus, the cam's slots can receive matched or unmatched cams, in particular in its range of heights or geometry can be combined in different ways. Therefore, surgeons have through the present invention a new set of structures and mechanisms to adjust angle aperture in frontal, sagittal, or frontal and sagittal planes simultaneously.

By rotating different combinations of cams, surgeons can correct bone angle in the frontal plane, in the sagittal plane or in frontal and sagittal planes simultaneously. The multiplicity of cams' possible combinations ranges of sizes and available heights may turn osteotomies more effective and subject to a lower number of fails and complications.

Referring now to FIGS. 7A-C there is shown three views of a locking member 100 or locking lid of the present invention; a perspective view (FIG. 7A), an anterior view (FIG. 7B) and a posterior view (FIG. 7C). In addition, the hole 50 for the insertion of the locking screw is in FIG. 13. The locking member 100 or locking plate also includes grooved pins 14 having a structure that can mechanically engage the keyed interior surface of the opening 75 in each of the cams that is used to rotate the cams. The grooved pins 14 and the keyed structure of the cams engage each other thereby maintaining the correct cam positioning and fixing the cams at the rotated angular position. Referring now also to FIGS. 8A-C there is shown a perspective view of three cams 71a-c of different sizes.

The present invention allows anterior adjusting: when the surgeon cuts the bone to create a certain corrective angle, by inserting this implant the angle aperture can be determined once the implant is in the bone. Instead of having several different devices for several different cases this solution of using for example three different pieces, can cover a wide range of bone openings. In exemplary illustrative embodiments the three different pieces covers a range of bone opening from approximately 6 mm up to approximately 12.3 mm. Also, only four cortical fixating screws are necessary to fix the device or mechanism to the bone (either regular cortical or locking type screws).

In addition, adjusting angle range is done with a single standard tool. For example, a hexagon Allen Key standard (e.g., square key type). The heaxagonal key can be inserted into the keyed opening 75 provided in each snail cam 70 and engaged with the keyed inner surface. In this way, appropriate movement of the hex key causes the snail cam to rotate until the spiral surface of the snail cam contacts the bone. As indicated herein, the cut brim construction of the snail cam in combination with the slot's construction assures that the snail cam does not fall out during rotation. Due to the fact that the wedge's surface in contact with the bone is not horizontal but slightly tilted, this generally allows the contact between the device and bone to be on a full surface. In contrast, this doesn't seem to happen with the conventional current implants due to their horizontal contact surfaces.

This mechanism also allows for the correction of bone angle on two different planes, the frontal plane and the sagittal plane. The angle correction on the sagittal plane is achievable by regulating or controlling the two snail cams individually. With this system, the bone opening measurement is continuous (not discrete). Also, the surgeon can control the size of the opening for example so as to between approximately 6 mm and approximately 12.3 mm, by appropriately selecting two of the exemplary wedges (e.g., three exemplary wedges).

This device 10 allows an upper anchorage provided by the two top screws. In this way, the surgeon can initially fix the device and then adjust bone angle accordingly. The surgeon doesn't have to have an exact measurement for correction angle aperture before inserting the device. As long as the surgeon has an accurate initial prediction, the angled aperture may then be adjusted once the device has been inserted.

It is expected that this will cut out any testing time necessary to ascertain the correct angled aperture, and consequently shorten surgery time. This way it allows one to control tibial slope, which is known to influence range of motion and stability (particularly in ACL central pivot-deficient knees). Furthermore, changes in the sagittal plane after HTO can be responsible for redistributing tibiofemoral joint contact pressures onto the posterior tibial plateau which might be a cause of pain and premature clinical failure after medial opening wedge tibial osteotomy.

Such a surgical kit of the present invention in addition to including the above-described components of the device also can include a hex key tool or the like that can be used to fix or tighten the four main screws (e.g., regular cortical or locking type screws) so that the base member 10 is securely attached to the bone or bony structure. Such a kit also can include another hexagon Allen Key standard (square key type) tool that can be used to separately adjust the snail cams to the desired angular position. In addition, the kit can include a plate wire tool that enables the surgeon to insert the device into the bone whilst also enabling minimal initial bone opening. The kit may also include various tools and instrumentation required for traditional osteotomy procedures.

Although a preferred embodiment of the invention has been described using specific terms, such description is for illustrative purposes only, and it is to be understood that changes and variations may be made without departing from the spirit or scope of the following claims.

### INCORPORATION BY REFERENCE

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. The device kit of claim 14, further comprising:
a securing mechanism that is mechanically coupled to the base member and arranged to secure the base member to the bone or bony structure;
wherein the spiral member of at least one of the plurality of adjusting mechanisms comprises at least one first portion, where the at least one first portion is arranged so it extends from the base member into the cut so as to be proximal to a resultant inferior cut surface; and
wherein the at least one first portion is rotatable with respect to the base member and dimensioned so as to be capable of maintaining two opposing surfaces of the cut in fixed spaced relation when disposed in the cut.

2. The device of claim 1, wherein the adjusting mechanism comprises two first portions that are spaced form each other, each first portion being rotatable with respect to the base member and each first portion being dimensioned so as to be capable of maintaining two opposing surfaces of the cut in fixed spaced relation.

3. (canceled)

4. The device of claim 1, wherein each the adjusting mechanism first portion includes a spiral shaped member that is rotatable with respect to the base member and which is configured so that the radius of the spiral varies as a function of the arc of rotation and wherein the radius of the spiral is established so as to define a plurality of discrete radii as a function of the arc of rotation, where the radii is correlated to the separation of the cut surfaces.

5. The device of claim 1, wherein the base member is configured with a mating structure and the adjusting mechanism includes a complimentary mating structure that is arranged to mate with the base member mating structure so as to hold each adjusting mechanism portion in fixed relation with respect to the base member.

6. The device of claim 5, wherein the base member mating structure comprises a slot in the base member and the adjusting mechanism mating structure comprises a mating portion that is received in the base member slot.

7. The device of claim 6, wherein the adjusting portion mating portion is rotatably secured to the adjusting portion first portion.

8. The device of claim 1, wherein the base member includes a plurality of through apertures arranged so as to be located on either side of the cut and the securing mechanism includes a plurality of mechanical fasteners one for each through aperture, wherein one mechanical fastener passes through a respective through aperture and is secured to the bone or bony structure whereby the base member is secured to the bone or bony structure.

9. The device of claim 8, where in the base member includes a plurality of through apertures arranged so as to be disposed opposite one side of the cut.

10. The device of claim 9, where in the base member includes a plurality of through apertures arranged so as to be disposed opposite both sides of the cut.

11. The device of claim 1, wherein the device is configured and arranged so as to used in a high tibial osteotomy procedure and wherein the cut or resulting opening is wedge shaped.

12. The device of claim 2, wherein the each of the first portions includes a spiral shaped member that is rotatable with respect to the base member and which is configured so that the radius of the spiral varies as a function of the arc of rotation and wherein the radius of one spiral member varies differently as a function of the arc of rotation than the second spiral member, whereby the two spiral members provide the adjustment of the resultant wedge in 2 planes.

13. The device of claim 12, wherein each spiral shaped member comprises a spiral cam structure.

14. A device kit for aligning a bone or bony structure using an osteotomy technique in one or both of a frontal and/or sagittal planes, said device kit comprising:
a base member extending in at least two directions, one direction being set so the base member spans a cut in the bone or bony structure;
a plurality of adjusting mechanisms, each being arranged so as to be separately mechanically coupled to the base member, each including a spiral member that is arranged so as to extend from the base member into the cut;
wherein each spiral members is rotatable with respect to the base member and is configured so that the radius of the spiral varies as a function of the arc of rotation and so as to be capable of maintaining two opposing surfaces of the cut in fixed spaced relation when disposed in the cut; and
wherein the radius of one of the plurality of spiral member varies differently as a function of the arc of rotation than another of the plurality of spiral members.

15. The device kit of claim 14, wherein the device kit further includes 3 or more adjusting mechanism with 3 or more spiral members, wherein at least one of the spiral members is configured so the radius of one of the three or more spiral member varies differently as a function of the arc of rotation than other 2 spiral members.

16. The device kit of claim 14, wherein the device kit includes a plurality of pairs of adjusting mechanism wherein the spiral members for one pair of the adjusting mechanisms are configured so the radius of said one pair member varies differently as a function of the arc of rotation for the spiral members of the other pair of plurality of adjusting mechanisms.

17-22. (canceled)
